(19) United States
(12) Patent Application Publication
(10) Pub. No.: US 2014/0105846 A1
(43) Pub. Date: Apr. 17, 2014

(54) COSMETIC POLYURETHANEUREA COPOLYMER COMPOSITION

(71) Applicants: Sophie Viala, Koln (DE); Sebastian Dörr, Duesseldorf (DE); Martin Huettner, Leverkusen (DE)

(72) Inventors: Sophie Viala, Koln (DE); Sebastian Dörr, Duesseldorf (DE); Martin Huettner, Leverkusen (DE)

(73) Assignee: BAYER INTELLECTUAL PROPERTY GMBH, Monheim (DE)

(21) Appl. No.: 14/006,026

(22) PCT Filed: Mar. 21, 2012
(86) PCT No.: PCT/EP12/54974

§ 371 Date: Dec. 23, 2013

(30) Foreign Application Priority Data
Mar. 25, 2011 (EP) 11159698.7

(51) Int. Cl. A61K 8/87; A61K 8/81; A61Q 5/06

(52) U.S. Cl. CPC A61K 8/87; A61Q 5/06; A61K 8/8152; A61K 8/8158
USPC 424/70.16

(57) ABSTRACT

The present invention relates to a cosmetic polyurethaneurea copolymer composition comprising a solvent, a polyurethaneurea and a copolymer, said polyurethaneurea being obtainable by reacting a single polyisocyanate component containing at least 75 mol % of IPDI, at least one polymeric polyol component, at least one hydrophilizing component and a single diamine component containing at least 75 mol % of IPDA, and said copolymer being obtainable by reaction of at least one monomer including acrylate groups with at least one further monomer. The invention further provides for the use of the cosmetic composition in cosmetics and a method for shaping hairstyles using the cosmetic composition.

The present invention relates to a cosmetic polyurethaneurea copolymer composition which can be used in particular in the field of hair cosmetics. Further subjects of the invention are the use of the cosmetic composition in cosmetics and a method for shaping hairstyles using the cosmetic composition.

Diverse hairstyles are created and stabilized using products which are known as hair-setting compositions. Hair-setting compositions come in most cases in the form of mousse setting compositions or hairsprays, differing little in their composition. Mousse setting compositions are applied to wet hair as aids for modeling the hairstyle. In contrast to this, hairsprays are applied to dry ready-styled hair to fix the hairstyle.

In the case of hairsprays and mousse setting compositions, the compositions for fixing or creating the hairstyle are usually in the form of preparations that can be sprayed from aerosol containers, squeezy bottles or by a pump, spraying or foaming devices, said preparations consisting of an alcoholic or aqueous-alcoholic solution of film-forming natural or synthetic polymers. These polymers can be selected from the group of nonionic, cationic, amphoteric or anionic polymers.

The film-forming polymers used in the prior art are often anionic or amphoteric polymers based on acrylates. However, the use of polyurethanes and polyurethaneureas as film formers is also known. Thus, hair setting compositions are described for example in WO 2009/118105 A1 which obtain a polyurethaneurea which is obtainable by reacting a water-insoluble non-water-dispersible isocyanate-functional polyurethane prepolymer with an amino-functional compound. The hair setting compositions disclosed here are highly suitable for fixing and stabilizing hair in the hairstyle desired in each case. However, it would be desirable if the hair was to simultaneously have higher elasticity in the fixed state.

It was therefore an object of the present invention to provide a cosmetic composition with the help of which hair can be fixed securely while retaining elasticity.

This object is achieved according to the invention by a cosmetic composition which comprises a solvent, a polyurethaneurea and a copolymer, where the polyurethaneurea is obtainable by reacting
a) a single polyisocyanate component containing at least 75 mol % isophorone diisocyanate (IPDI),
b) at least one polymeric polyol component,
c) at least one hydrophilizing component and
d) a single diamine component containing at least 75 mol % isophoronediamine (IPDA),
   and the copolymer is obtainable by reacting at least one monomer having acrylate groups with at least one further monomer.

Surprisingly, it has been found that hair which has been treated with the composition according to the invention has high strength and great elasticity. This was confirmed by flexural rigidity and omega-loop measurements.

According to the invention, an amino-functional chain extender component is understood as meaning a component which comprises at least one compound with two isocyanate-reactive amino groups and no hydrophilizing groups.

According to a first preferred embodiment of the composition according to the invention, the polyisocyanate component a) can comprise ≧80 mol %, preferably ≧85 mol %, further preferably ≧95 mol % and particularly preferably 100 mol %, IPDI.

Further polyisocyanates of component a) that can be used in addition to IPDI in a molar fraction of less than 25 mol %, are the aromatic, araliphatic, aliphatic or cycloaliphatic polyisocyanates with an NCO functionality of ≧2 that are known per se to the person skilled in the art.

Examples of such polyisocyanates are 1,4-butylene diisocyanate, 1,6-hexamethylene diisocyanate (HDI), 2,2,4 and/or 2,4,4-trimethylhexamethylene diisocyanate, the isomeric bis(4,4′-isocyanato-cyclohexyl)methanes or their mixtures of any desired isomer content, 1,4-cyclohexylene diisocyanate, 1,4-phenylene diisocyanate, 2,4- and/or 2,6-tolylene diisocyanate, 1,5-naphthylene diisocyanate, 2,2′- and/or 2,4′- and/or 4,4′-diphenylmethane diisocyanate, 1,3- and/or 1,4-bis(2-isocyanatoprop-2-yl)benzene (TMXDI), 1,3-bis(isocyanatomethyl)benzene (XDI), alkyl 2,6-diiso-cyanatohexanoate (lysine diisocyanates) with C1-C8-alkyl groups, and 4-isocyanatomethyl 1,8-octanediisocyanate (nonane triisocyanate) and triphenylmethane 4,4′,4″-triisocyanate.

As well as the aforementioned polyisocyanates, it is also possible to co-use in part modified diisocyanates or triisocyanates with a uretdione, isocyanurate, urethane, allophanate, biuret, iminooxadiazinedione and/or oxadiazinetrione structure.

They are preferably polyisocyanates or polyisocyanate mixtures of the type specified above with exclusively aliphatically and/or cycloaliphatically bonded isocyanate groups and an average NCO functionality of the mixture of 2 to 4, preferably of 2 to 2.6 and particularly preferably of 2 to 2.4.

If further polyisocyanates are used in component a) besides IPDI, particular preference is given to using 1,6-hexamethylene diisocyanate, the isomeric bis(4,4′-isocyanatocyclohexyl)methanes, and mixtures thereof.

It is likewise preferred if the polymeric polyol component b) has a number-average molecular weights of ≧400 to ≦8000 g/mol, particularly preferably from 600 to 3000 g/mol and/or an average OH functionalities of 1.5 to 6, preferably from 1.8 to 3 and particularly preferably from 1.9 to 2.1.

Possible constituents of the polymeric polyol component b) are the polyester polyols, polyacrylate polyols, polyurethane polyols, polycarbonate polyols, polyether polyols, polyester polyacrylate polyols, polyurethane polyacrylate polyols, polyurethane polyester polyols, polyurethane polyether polyols, polyurethane polycarbonate polyols and polyester polycarbonate polyols known per se in polyurethane coating technology. These can be used in b) individually or in any desired mixtures with one another.

Polyester polyols are for example the polycondensates, known per se, of di- and optionally tri-, and tetraols and di- and optionally tri- and tetracarboxylic acids or hydroxycarboxylic acids or lactones. Instead of the free polycarboxylic acids, the corresponding polycarboxylic anhydrides or corresponding polycarboxylic esters of lower alcohols can also be used for producing the polyesters.

Examples of diols suitable for this are ethylene glycol, butylene glycol, diethylene glycol, triethylene glycol, polyalkylene glycols, such as polyethylene glycol, also 1,2-propanediol, 1,3-propanediol, butanediol(1,3), butanediol(1,4), hexanediol(1,6) and isomers, neopentyl glycol or neopentyl glycol hydroxypivalate, with hexanediol(1,6) and isomers, neopentyl glycol and neopentyl glycol hydroxypivalate being preferred. In addition, polyols such as trimethylolpropane, glycerol, erythritol, pentaerythritol, trimethylolbenzene or trishydroxyethyl isocyanurate can also be used.

Dicarboxylic acids which can be used are phthalic acid, isophthalic acid, terephthalic acid, tetra-hydrophthalic acid, hexahydrophthalic acid, cyclohexanedicarboxylic acid, adipic acid, azelaic acid, sebacic acid, glutaric acid, tetrachlorophthalic acid, maleic acid, fumaric acid, itaconic acid, malonic acid, suberic acid, 2-methylsuccinic acid, 3,3-diethylglutaric acid and/or 2,2-dimethyl-succinic acid. The corresponding anhydrides can also be used as acid source.

If the average functionality of the polyol to be esterified is greater than 2, it is also possible to additionally co-use monocarboxylic acids, such as benzoic acid and hexanecarboxylic acid.

Preferred acids are aliphatic or aromatic acids of the aforementioned type. Particular preference is given to adipic acid, isophthalic acid and optionally trimellitic acid, very particularly preferably adipic acid.

Hydroxycarboxylic acids which can be co-used as reactants in the production of a polyester polyol with terminal hydroxyl groups are for example hydroxycaproic acid, hydroxybutyric acid, hydroxydecanoic acid, hydroxystearic acid and the like. Suitable lactones are caprolactone, butyrolactone and homologs. Preference is given to caprolactone.

It is likewise advantageous if the polymeric polyol component b) comprises a polyester, preferably a polyester based on adipic acid, or consists thereof.

In component b), it is also possible to use polycarbonates having hydroxyl groups, preferably polycarbonate diols, having number-average molecular weights of 400 to 8000 g/mol, preferably from 600 to 3000 g/mol. These are obtainable by reaction of carbonic acid derivatives, such as diphenyl carbonate, dimethyl carbonate or phosgene, with polyols, preferably diols.

Examples of suitable diols are ethylene glycol, 1,2- and 1,3-propanediol, 1,3- and 1,4-butanediol, 1,6-hexanediol, 1,8-octanediol, neopentyl glycol, 1,4-bishydroxymethylcyclohexane, 2-methyl-1,3-propanediol, 2,2,4-trimethylpentanediol-1,3, dipropylene glycol, polypropylene glycols, dibutylene glycol, polybutylene glycols, bisphenol A and lactone-modified diols of the aforementioned type. The polycarbonates having hydroxyl groups are preferably linear in structure.

Polyether polyols can likewise be used in component b). Of suitability are for example the polytetramethylene glycol polyethers known per se in polyurethane chemistry, as are obtainable by polymerization of tetrahydrofuran by means of cationic ring opening.

Further suitable polyether polyols are the addition products, known per se, of styrene oxide, ethylene oxide, propylene oxide, butylene oxides and/or epichlorohydrin onto di- or polyfunctional starter molecules.

Starter molecules that can be used are all compounds known according to the prior art, such as, for example, water, butyl diglycol, glycerol, diethylene glycol, trimethylolpropane, propylene glycol, sorbitol, ethylenediamine, triethanolamine, 1,4-butanediol. Preferred starter molecules are water, ethylene glycol, propylene glycol, 1,4-butanediol, diethylene glycol and butyl diglycol.

Suitable anionically or potentially anionically hydrophilizing compounds of component c) are compounds which have at least one isocyanate-reactive group such as a hydroxyl group or amino group and at least one functionality such as e.g. —COO-M⁺, —SO₃-M⁺, —PO(O-M⁺)₂ where M⁺ is for example metal cation, H⁺, NH₄⁺, NHR₃⁺, where R can in each case be a C1-C12-alkyl radical, C5-C6-cycloalkyl radical and/or a C2-C4-hydroxyalkyl radical, which enters into a pH-dependent dissociation equilibrium upon interaction with aqueous media and, in so doing, can be negatively or neutrally charged. Suitable anionically or potentially anionically hydrophilizing compounds are mono- and dihydroxycarboxylic acids, mono- and dihydroxysulfonic acids, and also mono- and dihydroxyphosphonic acids and their salts. Examples of such anionic or potentially anionic hydrophilizing agents are dimethylolpropionic acid, dimethylolbutyric acid, hydroxypivalic acid, malic acid, citric acid, glycolic acid, lactic acid and the propoxylated adduct of 2-butenediol and NaHSO₃, as is described in DE-A 2 446 440, pages 5-9, formula I-III. Particularly preferred anionic or potentially anionic hydrophilizing agents of component c) are those of the aforementioned type which have carboxylate or carboxylic acid groups and/or sulfonate groups.

According to a further advantageous embodiment of the composition according to the invention, it is provided that the hydrophilizing component c) is an anionically hydrophilizing component and preferably a sulfonate.

Suitable nonionically hydrophilizing compounds of component c) are e.g. polyoxyalkylene ethers which contain at least one hydroxy or amino group, preferably at least one hydroxy group.

Examples are the monohydroxy-functional polyalkylene oxide polyether alcohols having on statistical average 5 to 70, preferably 7 to 55, ethylene oxide units per molecule, which are accessible in a manner known per se by alkoxylation of suitable starter molecules (described e.g. in Ullmanns Encyclopädie der technischen Chemie [Ullmanns Encyclopedia of Industrial Chemistry], 4th edition, volume 19, Verlag Chemie, Weinheim pp. 31-38). These compounds are either pure polyethylene oxide ethers or mixed polyalkylene oxide ethers, in which case, however, they then contain at least 30 mol %, preferably at least 40 mol %, based on all contained alkylene oxide units, of ethylene oxide units. Particularly preferred nonionic compounds are monofunctional, mixed polyalkylene oxide polyethers which have 40 to 100 mol % ethylene oxide units and 0 to 60 mol % propylene oxide units.

For the hydrophilization it is also possible to use mixtures of anionic or potentially anionic hydrophilizing agents and nonionic hydrophilizing agents.

In a development of the invention, it is provided that the amino-functional chain extender component d) can comprise ≧85 mol %, preferably ≧95 mol % and particularly preferably 100 mol %, IPDA.

As further constituents of the amino-functional chain extender d), further NH₂- and/or NH-functional compounds can be used besides IPDI.

Suitable components—which can be used in addition to IPDA in a molar fraction of less than 25%—are di- or polyamines such as 1,2-ethylenediamine, 1,2- and 1,3-diaminopropane, 1,4-diamino-butane, 1,6-diaminohexane, 2,2,4- and 2,4,4-trimethylhexamethylenediamine, 2-methylpenta-methylenediamine, diethylenetriamine, triaminononane, 1,3- and 1,4-xylylenediamine, α,α,α′,α′-tetramethyl-1,3- and -1,4-xylylenediamine and 4,4-diaminodicyclohexylmethane and/or dimethylethylenediamine. Hydrazine or and hydrazides such as adipic dihydrazide are likewise possible.

Preferably, the chain extension/termination is carried out before dispersion in water, the isocyanate groups reacting with the chain extender to give urea groups.

Apart from components a) to d), further building blocks can also be used for producing the polyurethaneurea.

Examples of further building blocks are hydroxy-functional compounds with molecular weights of 62 to 399 g/mol, such as, for example, polyols of the specific molecular weight range having up to 20 carbon atoms, such as ethylene glycol, diethylene glycol, triethylene glycol, 1,2-propanediol, 1,3-propanediol, 1,4-butanediol, 1,3-butylene glycol, cyclohexanediol, 1,4-cyclohexanedimethanol, 1,6-hexanediol, neopentyl glycol, hydroquinone dihydroxyethyl ether, bisphenol A (2,2-bis(4-hydroxyphenyl)propane), hydrogenated bisphenol A, (2,2-bis(4-hydroxycyclohexyl)propane), trimethylolpropane, glycerol, pentaerythritol. Furthermore, monofunctional isocyanate-reactive amine compounds can be used, such as, for example, methylamine, ethylamine, propylamine, butylamine, octylamine, laurylamine, stearylamine, isononyloxypropylamine, dimethylamine, diethylamine, dipropylamine, dibutylamine, n-methylaminopropylamine, diethyl(methyl)-aminopropylamine, morpholine, piperidine.

However, apart from components a) to d), no further building blocks are preferably used for producing the polyurethaneurea according to the invention.

The preparation of the polyurethaneurea can take place by processes known to the person skilled in the art in one or more stage(s) in homogeneous or for multistage reaction and be carried out in part in disperse phase. A dispersing, emulsifying or dissolution step preferably takes place following completely or partially carried out polyaddition from a) to d). Subsequently, a further polyaddition or modification optionally takes place in disperse or dissolved (homogeneous) phase. In this connection, it is possible to use all processes known from the prior art, such as e.g. prepolymer mixing processes, acetone processes or melt dispersion processes. Preference is given to working in accordance with the acetone process.

The copolymer present in the composition according to the invention is obtainable by reacting at least one monomer having acrylate groups with at least one further monomer.

Suitable monomers having acrylate groups are in particular methyl acrylate, ethyl acrylate, propyl acrylate, isopropyl acrylate, n-butyl acrylate, sec-butyl acrylate, tert-butyl acrylate, pentyl acrylate, hexyl acrylate, heptyl acrylate, octyl acrylate, 2-octyl acrylate, ethylhexyl acrylate, nonyl acrylate, 2-methyloctyl acrylate, 2-tert-butylheptyl acrylate, 3-isopropylheptyl acrylate, decyl acrylate, undecyl acrylate, 5-methylundecyl acrylate, dodecyl acrylate, 2-methyldodecyl acrylate, tridecyl acrylate, 5-methyltridecyl acrylate, tetradecyl acrylate, pentadecyl acrylate, hexadecyl acrylate, 2-methylhexadecyl acrylate, heptadecyl acrylate, 5-isopropylheptadecyl acrylate, 5-ethyloctadecyl acrylate, octadecyl acrylate, nonadecyl acrylate, eicosyl acrylate, cycloalkyl acrylates, such as, for example, cyclopentyl acrylate, cyclohexyl acrylate, 3-vinyl-2-butylcyclohexyl acrylate, cycloheptyl acrylate, cyclooctyl acrylate, bornyl acrylate, tetrahydrofurfuryl acrylate and isobornyl acrylate. Preference is given to ethyl acrylate, n-butyl acrylate, ethylhexyl acrylate, cyclohexyl acrylate and particular preference is given to ethyl acrylate, n-butyl acrylate or ethylhexyl acrylate.

The further monomer is advantageously selected from the group of nonionic, anionic, amphoteric and/or cationic monomer and mixtures thereof.

Particularly suitable further monomers, which may be present on their own or in mixtures, preferably also with anionic and/or amphoteric and/or zwitterionic monomers, are:
styrene or derivatives thereof
vinyl acetate
acrylic acid or methacrylic acid or salts thereof. These include for example copolymers of vinylpyrrolidones, acrylic acid and C1-C20 alkyl methacrylates, e.g. Acrylidone® LM from ISP, copolymers of methacrylic acid, ethyl acrylates and tert-butyl acrylates, e.g. Luvimer® 100 P from BASF, ethyl acrylate/N-tert-butylacrylamide/acrylic acid copolymers ULTRAHOLD® STRONG from BASF;
crotonic acid derivative or salts thereof. These include for example vinyl acetate/crotonic acid, vinyl acetate/acrylate and/or vinyl acetate/vinyl neodecanoate/crotonic acid copolymers, e.g. Resyn® 28-1310 or Resyn® 28-2930 from AkzoNobel or Luviset® CAN from BASF, sodium acrylate/vinyl alcohol copolymers;
unsaturated C4-C8 carboxylic acid derivatives or carboxylic anhydride copolymers selected from copolymers of maleic acid or maleic anhydride or fumaric acid or fumaric anhydride or itaconic acid or itaconic anhydride and at least one acrylic acid ester. Very particularly advantageous polymers are copolymers of methyl vinyl ether and maleic anhydride partly esterified, e.g. GANTREZ®;
acrylamides or methacrylamides. These include for example acrylates/octylacrylamide copolymer, e.g. Amphomer® HC from AkzoNobel;
basic monomers which are derived from a substituted vinyl compound with at least one basic atom, such as in particular dialkylaminoalkyl methacrylate and acrylate, dialkylaminoalkylmethacrylamide and -acrylamide. Preferred basic comonomers are aminoethyl methacrylate, butylaminoethyl methacrylate, N,N-dimethylaminoethyl methacrylate, N-t-butylaminoethyl methacrylate. Very particularly advantageous amphoteric polymers are e.g. the copolymers octylacrylamide/acrylates/butylaminoethyl methacrylate copolymer, which are commercially available under the names AMPHOMER®, AMPHOMER® LV 71 or BALANCE® 47 from AkzoNobel, and methyl methacrylate/methyldimethylcarboxymethylammonium ethyl methacrylate copolymers.

It is very particularly preferred if the copolymer comprises octylacrylamide/acrylates/butylaminoethyl methacrylate copolymer or consists thereof.

The solvent can comprise cosmetically suitable solvents. Preferred solvents are aliphatic alcohols with C2-4 carbon atoms, such as isopropanol, t-butanol, n-butanol; polyols such as propylene glycol, glycerol, ethylene glycol and polyol ethers; acetone; unbranched or branched hydrocarbons such as pentane, hexane, isopentane and cyclic hydrocarbons such as cyclopentane and cyclohexane; and mixtures thereof.

It is particularly preferred if the solvent comprises ethanol. Its content in the solvent can be ≧10% by weight and ≦95% by weight, preferably ≧15% by weight and ≦90% by weight, further preferably ≧18% by weight and ≦60% by weight and particularly preferably ≧20% by weight and 60% by weight.

It is likewise possible for the solvent to comprise water. Preferably, its content content in the solvent can be ≧0% by weight and ≦50% by weight, further preferably ≧0% by weight and ≦40% by weight, yet further preferably ≧10% by weight and ≦40% by weight and particularly preferably ≧20% by weight and ≦40% by weight.

It is particularly preferred if the solvent comprises ethanol and water or consists thereof.

Preference is also given to a composition which comprises ≧10% by weight and ≦98% by weight, preferably ≧20% by weight and ≦98% by weight, further preferably ≧30% by weight and ≦98% by weight and particularly preferably ≧40% by weight and ≦98% by weight, of the solvent.

According to a further embodiment of the composition according to the invention, it is provided that it comprises ≧0.1% by weight and ≦30% by weight, preferably ≧0.1% by weight and ≦20% by weight, further preferably ≧0.5% by weight and ≦15% by weight and particularly preferably ≧0.5% by weight and ≦10% by weight, of the polyurethaneurea.

It is likewise preferred if the composition comprises ≧0.1% by weight and ≦15.0% by weight, preferably ≧0.2% by weight and ≦10.0% by weight, further preferably ≧0.5% by weight and ≦8.0% by weight and particularly preferably ≧1.0% by weight and ≦6.0% by weight, of the copolymer.

Besides the polyurethaneurea described above and the copolymer, the composition according to the invention can comprise further suitable film formers which can contribute in particular also to the setting and the styling of the hair.

The fraction of further film formers can be from 0 to 20% by weight and in particular 0 to 10% by weight, based on the total composition.

The further film former(s) are advantageously selected from the group of nonionic, anionic, amphoteric and/or cationic polymers and mixtures.

The composition according to the invention can furthermore comprise thickeners. Advantageous thickeners are:
Crosslinked acrylic acid or methacrylic acid homo- or copolymers. These include crosslinked homopolymers of methacrylic acid or acrylic acid, copolymers of acrylic acid and/or methacrylic acid and monomers which are derived from other acrylic or vinyl monomers, such as C10-30 alkyl acrylates, C10-30-alkyl methacrylates and vinyl acetate.
Thickening polymers of natural origin, for example based on cellulose, guar gum, xanthan, scleroglucan, gellan gum, rhamsan and karaya gum, alginates, maltodextrin, starch and its derivatives, carob seed flour, hyaluronic acid.
Nonionic, anionic, cationic or amphoteric associative polymers e.g. based on polyethylene glycols and their derivatives, or polyurethanes.
Crosslinked or non-crosslinked homopolymers or copolymers based on acrylamide or methacrylamide, such as homopolymers of 2-acrylamido-2-methylpropanesulfonic acid, copolymers of acrylamide or methacrylamide and methacryloyloxyethyltrimethyl-ammonium chloride or copolymers of acrylamide and 2-acrylamido-2-methylpropane-sulfonic acid.

Particularly advantageous thickeners are thickening polymers of natural origin, crosslinked acrylic acid or methacrylic acid homo- or copolymers and crosslinked copolymers of 2-acrylamido-2-methylpropanesulfonic acid.

Preference is likewise given to xanthan gum such as the products supplied under the names Keltrol® and Kelza® by CP Kelco, or the products from RHODIA Rhodopol and guar gum which are supplied under the name Jaguar® HP105.

Further thickeners are crosslinked homopolymers of methacrylic acid or acrylic acid which are commercially available from Lubrizol under the names Carbopol® 940, Carbopol® 941, Carbopol® 980, Carbopol® 981, Carbopol® ETD 2001, Carbopol® EDT 2050, Carbopol® 2984, Carbopol® 5984 and Carbopol® Ultrez 10, from 3V under the names Synthalen® K, Synthalen® L and Synthalen® MS and from PROTEX under the names Modarez® V 1250 PX, Modarez® V2000 PX, Viscaron® A1600 PE and Viscaron® A700 PE.

Likewise preferred thickeners are crosslinked copolymers of acrylic acid or methacrylic acid and a C₁₀₋₃₀-alkyl acrylate or C₁₀₋₃₀-alkyl methacrylate and copolymers of acrylic acid or methacrylic acid and vinylpyrrolidone. Such copolymers are commercially available for example from Lubrizol under the names Carbopol® 1342, Carbopol® 1382, Pemulen® TR1 or Pemulen® TR2 and from ISP under the names Ultrathix P-100 (INCI: Acrylic Acid/VP Crosspolymer).

Very particularly advantageous thickeners are crosslinked copolymers of 2-acrylamido-2-methyl-propanesulfonic acid. Such copolymers are available for example from Clariant under the names Aristoflex® AVC (INCI: Ammonium Acryloyldimethyltaurate/VP Copolymer).

If thickeners are used, they are generally present in the composition in a concentration of 0% to 2% by weight, preferably 0% to 1% by weight.

The composition according to the invention can furthermore comprise a propellant gas.

Preferred propellant gases are hydrocarbons such as propane, isobutane and n-butane, and mixtures thereof. Compressed air, carbon dioxide, nitrogen, nitrogen dioxide and dimethyl ether, as well as mixtures of all of these gases can likewise be used.

The person skilled in the art is naturally aware that there are propellant gases that are nontoxic per se which would in principle be suitable for realizing the present invention in the form of aerosol preparations but which nevertheless should be dispensed with on account of an unacceptable impact on the environment or other accompanying phenomena. These are in particular fluorocarbons and chlorofluorocarbons (CFCs) such as e.g. 1,2-difluoroethane (propellant 152 A).

Furthermore, hair care active ingredients may be present in the composition according to the invention. Care substances which can preferably be used are cyclic polydimethylsiloxanes (cyclomethicones) or silicone surfactants (polyether-modified siloxanes) of the dimethicone copolyol type or simethicone type. Cyclomethicones are supplied inter alia under the trade names Abil® K4 by Goldschmidt or e.g. DC 244, DC 245 or DC 345 by Dow Corning. Dimethicone copolyols are supplied e.g. under the trade name DC 193 by Dow Corning or Belsil® DM 6031 by Wacker.

Optionally, conventional additives can be present in the composition, for example in order to impart certain modifying properties to it. These may be for example silicones or silicone derivatives, wetting agents, humectants, softeners such as glycerol, glycol and phthalic esters and ethers, fragrances and perfumes, UV absorbers, dyes, pigments, and other colorants, anticorrosive agents, neutralizing agents, antioxidants, anti-sticking agents, combining agents and conditioning agents, antistatic agents, shine agents, preservatives, proteins and derivatives thereof, amino acids, vitamins, emulsifiers, surface-active agents, viscosity modifiers, thickeners and rheology modifiers, gelling agents, opacifiers, stabilizers, surfactants, sequestrants, complexing agents, pearlizing agents, esthetic boosters, fatty acids, fatty alcohols, triglycerides, botanical extracts, clarifying auxiliaries and film formers.

These additives are present in the composition generally in a concentration of from about 0.001% to 15% by weight, preferably 0.01% to 10% by weight.

The composition according to the invention can advantageously be present in a pump spray or aerosol packaging. It can also be foamed using a propellant gas.

A further preferred embodiment of the composition according to the invention is in the form of a spray which additionally comprises one or more of the following constituents: cosmetically suitable solvents, such as aliphatic alcohols with 2-4 carbon atoms, preferably ethanol, polyols, acetone, unbranched or branched hydrocarbons, cyclic hydrocarbons and mixtures thereof, and also propellant gases such as hydrocarbons, compressed air, carbon dioxide, nitrogen, nitrogen dioxide, dimethyl ether, fluorocarbons and chlorofluorocarbons, preferably dimethyl ether and/or a propane/butane mixture.

The composition according to the invention can, however, also be in the form of a foam, a gel, an emulsion, a solution or a cream.

The present invention further provides the use of a composition according to the invention in cosmetics, preferably in the field of hair cosmetics, particularly preferably in the field of hairstyling.

The invention also provides the use of a cosmetic composition according to the invention in cosmetics, preferably in the field of hair cosmetics, particularly preferably in the field of hairstyling.

The present invention yet further provides a method for shaping hairstyles in which a cosmetic composition according to the invention is applied to hair.

The present invention is illustrated below by reference to the following examples. Unless stated otherwise, all quantitative data, fractions and percentages are based on the weight and the total amount or on the total weight of the composition.

### EXAMPLES

Unless noted otherwise, all analytical measurements refer to measurements at temperatures of 23° C.

The solid contents or solid-body contents were ascertained in accordance with DIN EN ISO 3251 by heating a weighed sample at 105° C. to constant weight. At constant weight, the solid-body content was calculated by reweighing the sample.

Unless expressly mentioned otherwise, NCO values were determined volumetrically in accordance with DIN-EN ISO 11909.

The monitoring of free NCO groups was carried out by means of IR spectroscopy (band at 2260 cm⁻¹).

The stated viscosities were determined by means of rotary viscometry in accordance with DIN 53019 at 23° C. using a rotary viscometer from Anton Paar Germany GmbH, Ostfildern, Germany.

Determination of the average particle sizes (the number average is stated) of the polyurethane dispersions was carried out after dilution with deionized water by means of laser correlation spectroscopy (instrument: Malvern Zetasizer 1000, Malver Inst. Limited).

### Substances Used and Abbreviations:

Diaminosulfonate: NH₂—CH₂CH₂—NH—CH₂CH₂—SO₃Na (45% strength in water)

### Example According to the Invention

### Polyurethaneurea Dispersion A

1360.0 g of a polyester of adipic acid, hexanediol and neopentyl glycol with an average molecular weight of 1700 g/mol were heated to 65° C. Then, 318.5 g of isophorone diisocyanate (IPDI) were added and the mixture was stirred at 105° C. until the NCO value was below the theoretical value. The finished prepolymer was dissolved with 3000 g of acetone at 50° C. and then a solution of 23.4 g of isophoronediamine (IPDA), 129.6 g of diaminosulfonate and 357 g of water was metered in. The after-stirring time was 15 min. The mixture was then dispersed by adding 2900 g of water. The solvent was removed by distillation in vacuo and a storage-stable dispersion was obtained, the solids content being adjusted by adding water.

Solids content: 32%
Particle size (LCS): 27 nm
Viscosity: 1500 mPas
pH: 7.3

### Example According to the Invention

### Polyurethaneurea Dispersion B

318.8 g of a polyester of adipic acid, hexanediol and neopentyl glycol with an average molecular weight of 1700 g/mol were heated to 65° C. Then, 70.9 g of isophorone diisocyanate were added and the mixture was stirred at 105° C. until the NCO value was below the theoretical value. The finished prepolymer was dissolved with 700 g of acetone at 50° C. and then a solution of 3.5 g of isophoronediamine, 30.4 g of diaminosulfonate and 84 g of water was metered in. The after-stirring time was 15 min. The mixture was then dispersed by adding 513 g of water. The solvent was removed by distillation in vacuo and a storage-stable dispersion was obtained, the solids content being adjusted by adding water.

Solids content: 30%
Particle size (LCS): 32 nm
Viscosity: 1000 mPas
pH: 7.2

### Example According to the Invention

### Polyurethaneurea Dispersion C

319 g of a polyester of adipic acid, hexanediol and neopentyl glycol with an average molecular weight of 1700 g/mol were heated to 65° C. Then, 79.2 g of isophorone diisocyanate were added and the mixture was stirred at 105° C. until the NCO value was below the theoretical value. The finished prepolymer was dissolved with 1100 g of acetone at 50° C. and then a solution of 8.0 g of isophoronediamine, 30.4 g of diaminosulfonate and 84 g of water was metered in. The after-stirring time was 15 min. The mixture was then dispersed by adding 540 g of water. The solvent was removed by distillation in vacuo and a storage-stable dispersion was obtained, the solids content being adjusted by adding water.

Solids content: 28%
Particle size (LCS): 35 nm
Viscosity: 760 mPas
pH: 7.7

### Application-Related Comparative Experiments:

### Flexural Rigidity Measurement

For the flexural rigidity measurement, commercially available European mixed hair (useful length: 21 cm, weight: 2.4 g) was used. Prior to use, the hair was subjected to a standardized washing procedure. For this, the hair was softened in water for 15 minutes and then shampooed with 0.2 ml of standard shampoo for one minute, thoroughly rinsed with warm water, blow-dried on cold and conditioned at 21±1° C. and 50±5% relative humidity.

Individual hair tresses were then dipped into the respective cosmetic composition for two minutes. The hair tresses were then combed twice.

Overall, ca. 0.40 to 0.45 g of the polymer constituents of the compositions remained on the hair tress.

The “flexural rigidity measurement were carried out in a specially climatized chamber at a relative humidity of 50±5% using a universal testing machine (Zwick 1120, Zwick, Ulm). During the measurement, the maximum force was measured in order to bend the hair tress. The temperature in the chamber was 21±1° C. Each experiment was carried out with ten tresses.

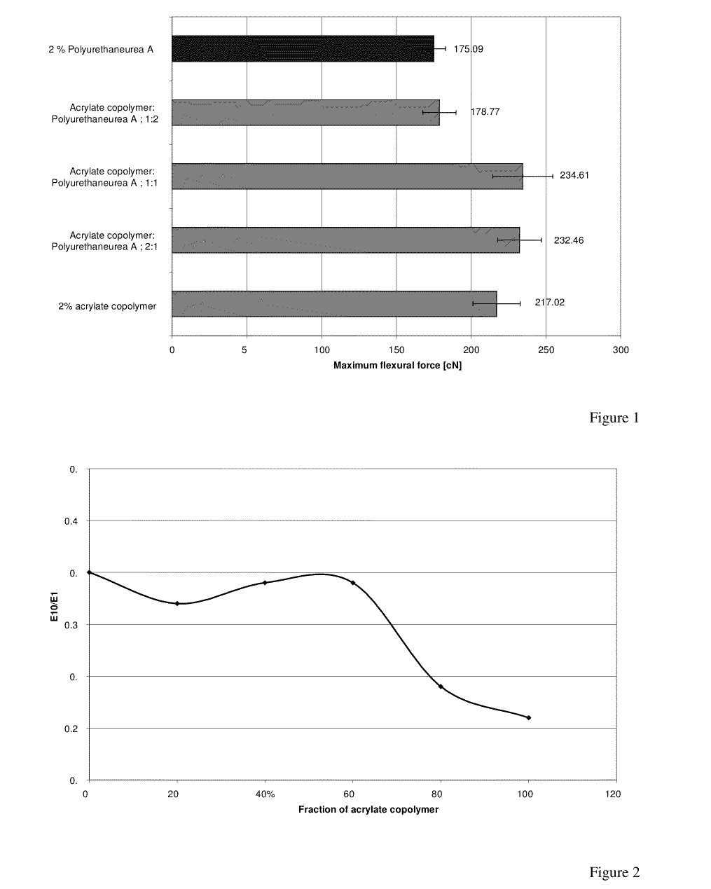

The results of the flexural rigidity measurements of compositions which comprise only a polyurethaneurea as polymeric constituents and compositions according to the invention are shown graphically in FIG. 1.

The measurements show that the compositions according to the invention bring about a higher maximum flexural force in the case of treated hair than is possible with compositions which comprise only a polyurethaneurea as polymeric component.

### Omega Loop Measurement:

For the omega loop measurement, commercially available hair mixtures (European hair, Chinese hair and fine European hair, weight: 0.2 g from International Hair Importers, New York) were used.

Prior to use, the hair was subjected to a standardized washing procedure. Then, in each case one hair tress is shaped into an “omega” and treated with 150 μg of the respective cosmetic composition (4% by weight active aqueous polymer solution). A Texture Analyzer (model TA-XT2 from Texture Technologies Corporation) was then used to measure the necessary force which was required in order to reduce the height of the hair tress by 25%. The measurement was repeated ten times. The “omega loop” experiments were carried out in a special climatized chamber at a relative humidity of 50±5%. The temperature in the chamber was 20±1° C. Each experiment was carried out with two tresses. The detailed method for carrying out the omega loop measurement is described in the article by Jachowicz, J. and McMullen, R. J. Cosmetic Science, 2002, 53, 345-361, to which reference is made in this respect.

The results of the omega loop measurements are shown in FIG. 2. The applied force is shown as a function of the change in height of the hair tress. The flexibility was defined as the ratio of the modulus after the tenth deformation (=increase in the curve) to the modulus after the first deformation (E10/E1). The higher E10/E1, the more flexible the polymer film on the hair.

FIG. 2 shows that the ratio E10/E1 increases with increasing fraction of polyurethaneurea in the composition. The combination of the polyurethaneurea with the acrylate copolymer thus leads to a more flexible polymer film on the hair. For a weight fraction of 60% of the acrylate copolymer in the composition, the flexibility is similar to that of a composition which comprises only the polyurethaneurea.

In summary, it remains to be emphasized that a composition according to the invention which comprises both a polyurethaneurea and an acrylate copolymer is suitable for fixing hair flexibly with a strong hold.

### Application Examples

Examples of cosmetic formulations are listed below in which the compositions according to the invention can be used. In each case, it is stated how many parts by weight of the individual components are present in a formulation.

Pump-setting spray

| | A | B |
|---|---|---|
| Polyurethaneurea according to | 2 | 5 |
| the invention (based on solids) | | |
| Acrylates/octylacrylamide | | 5 |
| copolymer¹ | | |
| Acrylates copolymer² | 1 | |
| Aminomethylpropanol | q.s. | q.s. |
| Ethanol | 90 | 55 |
| Perfume | q.s. | q.s. |
| Water | ad 100% | ad 100% |

q.s. quantum satis
¹Amphomer ® HC, AkzoNobel
²Luvimer ® 100 P, BASF

Aerosol hair sprays

| | C | D | E | G |
|---|---|---|---|---|
| Polyurethaneurea accord- | 2 | 7 | 2 | 6 |
| ing to the invention | | | | |
| (based on solids) | | | | |
| Octylacrylamide/acrylate/ | 3 | | | |
| butylaminoethyl meth- | | | | |
| acrylate³ (based on | | | | |
| solids) | | | | |
| Octylacrylamide/acrylate/ | | 3 | | 2.0 |
| butylaminoethyl meth- | | | | |
| acrylate⁴ (based on | | | | |
| solids) | | | | |
| Acrylate copolymer⁵ | | | 2 | |
| Aminomethylpropanol | q.s. | q.s. | q.s | q.s. |
| Glycerol | | 0.5 | | |
| Panthenol | | | 0.5 | 0.5 |
| PEG/PPG-18/18 | | | | |
| dimethicone | | | | |
| PEG-12 dimethicone | | 0.05 | | |
| Propylene glycol | | | | |
| Cyclomethicone | | | 1.0 | 1.0 |
| Benzophenone-3 | | 0.1 | 0.1 | 0.1 |
| Perfume | q.s | q.s | q.s | q.s |
| Ethanol | 30 | 25 | 70 | 50 |
| Water | ad 100% | ad 100% | ad 100% | ad 100% |
| Propane/butane 3.5 bar | | | 20 | |
| (20° C.) | | | | |
| Dimethyl ether | 40 | 30 | | |
| Fluorocarbon 152 A | | | | 20 |

q.s. quantum satis
³Amphomer ® LV-71, AkzoNobel
⁴Amphomer ®, AkzoNobel
⁵Luvimer ® P-100, BASF

Hair mousse

| | H | I |
|---|---|---|
| Polyurethaneurea according to the | 1 | 3 |
| invention (based on solids) | | |
| Octylacrylamide/acrylate/butylamino- | 1 | |
| ethyl methacrylate⁶ (based on solids) | | |
| Acrylates/t-butylacrylamide copolymer⁷ | | 1 |
| Glycerol | 0.1 | |
| Aminomethylpropanol | q.s. | q.s |
| Panthenol | 0.05 | 0.5 |
| Polyquaternium-164 | 2 | |
| Cetyltrimethylammonium chloride | 0.2 | 0.5 |
| PEG-12 dimethicone | | 0.5 |
| Cyclomethicone | | 0.5 |
| Benzophenone-3 | 0.1 | |
| Perfume | q.s. | q.s. |
| Ethanol | 5 | 10 |
| Water | ad 100% | ad 100% |
| Preservative | q.s. | q.s. |
| Propane/butane/dimethyl ether | 10 | 7 |
| Dimethyl ether | | 7 |
| Fluorocarbon 152 A | | 3 |

q.s. quantum satis
⁶Amphomer ®, AkzoNobel
⁷Ultrahold ® 8, BASF

Hair gel/cream

| | J | K | L |
|---|---|---|---|
| Polyurethaneurea according to the | 2 | 1 | 5 |
| invention (based on solids) | | | |
| Octylacrylamide/acrylate/butyl- | 3 | 1 | |
| aminoethyl methacrylate⁸ (based on | | | |
| solids) | | | |
| Acrylate copolymer⁹ | | | 3 |
| Carbomer | 0.8 | | |
| Acrylic acid acid/VP crosspolymer | | 0.5 | |
| Ammonium acryloyldimethyl- | | | 0.8 |
| taurate/VP copolymer | | | |
| Glycerol | 0.5 | | |
| Panthenol | | 0.5 | 0.5 |
| Propylene glycol | | | 0.2 |
| Cyclomethicone | | 0.2 | |
| Neutralizing agent | q.s. | q.s. | q.s. |
| Perfume | q.s. | q.s. | q.s. |
| Ethanol | 6 | | |
| Water | ad 100% | ad 100% | ad 100% |
| Preservative | q.s. | q.s. | q.s. |

q.s. quantum satis
⁸Amphomer ®, AkzoNobel
⁹Luvimer ® P-100, BASF

1. A cosmetic composition comprising a solvent, a polyurethaneurea and a copolymer, where said polyurethaneurea is obtainable by reacting
a) a single polyisocyanate component at least 75 mol % isophorone diisocyanate (IPDI),
b) at least one polymeric polyol component,
c) at least one hydrophilizing component and
d) a single diamine component comprising at least 75 mol % isophoronediamine (IPDA),
and said copolymer is obtainable by reacting at least one monomer having acrylate groups with at least one further monomer.

2. The composition as claimed in claim 1, wherein said polyisocyanate component a) comprises ≧80 mol %, optionally ≧85 mol %, further optionally 95 mol % or optionally 100 mol %, IPDI.

3. The composition as claimed in claim 1, wherein said polymeric polyol component b) has a number-average molecular weight of ≧400 to ≦8000 g/mol and/or an OH functionality functionalities of from 1.5 to 6.

4. The composition as claimed in claim 1, wherein said polymeric polyol component b) comprises a polyester, optionally a polyester based on adipic acid, or said polymeric polyol consists of a polyester.

5. The composition as claimed in claim 1, wherein said hydrophilizing component c) is an anionically hydrophilizing component and optionally a sulfonate.

6. The composition as claimed in claim 6, wherein said diamine component d) comprises ≧85 mol %, optionally ≧95 mol %, and optionally 100 mol %, IPDA.

7. The composition as claimed in claim 1, wherein said solvent comprises ethanol and optionally ≧10% by weight and ≦95% by weight, further optionally ≧15% by weight and ≦90% by weight, or optionally ≧18% by weight and ≦60% by weight or optionally ≧20% by weight and ≦60% by weight, of ethanol.

8. The composition as claimed in claim 1, the wherein said solvent comprises water and optionally ≧0% by weight and ≦50% by weight, further optionally ≧0% by weight and ≦40% by weight or optionally ≧10% by weight and ≦40% by weight or optionally ≧20% by weight and ≦40% by weight, of water.

9. The composition as claimed in claim 1, wherein said copolymer comprises octylacrylamide/acrylates/butylaminoethyl methacrylate copolymer or said copolymer consists of octylacrylamide/acrylates/butylaminoethyl methacrylate copolymer.

10. The composition as claimed in claim 1, wherein said composition comprises ≧0.1% by weight and ≦30% by weight, optionally ≧0.1% by weight and ≦20% by weight, further optionally ≧0.5% by weight and ≦15% by weight or optionally ≧0.5% by weight and ≦10% by weight, of the polyurethaneurea.

11. The composition as claimed in claim 1, wherein said composition comprises ≧0.1% by weight and ≦15.0% by weight, optionally ≧0.2% by weight and ≦10.0% by weight, further optionally ≧0.5% by weight and ≦8.0% by weight or optionally ≧1.0% by weight and ≦6.0% by weight, of the copolymer.

12. A composition as claimed in claim 1 capable of being used in a cosmetic, optionally in a field of hair cosmetics, or optionally in a field of hairstyling.

13. A method for shaping a hairstyle comprising applying a cosmetic composition as claimed in claim 1 to hair.
